(19) United States
(12) Patent Application Publication
(10) Pub. No.: US 2016/0038186 A1
(43) Pub. Date: Feb. 11, 2016

(54) DEVICES AND METHODS FOR BONE ANCHORING

(71) Applicant: CYCLA ORTHOPEDICS LTD., Bat-Shlomo (IL)

(72) Inventors: Rafi HERZOG, Bat-Shlomo (IL); Ofer VIKINSKY, Tzur-Yigal (IL)

(73) Assignee: Cycla Orthopedics Ltd., Bat-Shlomo (IL)

(21) Appl. No.: 14/774,151

(22) PCT Filed: Mar. 12, 2014
(86) PCT No.: PCT/IL14/50252

§ 371 Date: Sep. 10, 2015

Related U.S. Application Data
(60) Provisional application No. 61/792,279, filed on Mar. 15, 2013.
(60) Provisional application No. 61/835,648, filed on Jun. 17, 2013.

(51) Int. Cl. A61B 17/68; A61B 17/88; A61B 17/86; A61B 17/84

(52) U.S. Cl. CPC A61B 17/683; A61B 17/848; A61B 17/8869; A61B 17/685; A61B 17/686; A61B 17/864; A61B 2017/681

(57) ABSTRACT

A device for fixation of bone tissue and methods of using same are provided. The device includes a first anchor positionable within a first bone region, a second anchor positionable within a second bone region and a wire interconnecting the first and second anchors. The wire is attached to the first anchor via an elastically deformable element having a force constant (K) of 20-80 N/mm along a longitudinal axis of the first anchor.

### FIELD AND BACKGROUND OF THE INVENTION

The present invention relates to a bone anchoring system and methods of using same, and more particularly, to an anchor system which can be used to interconnect adjacent bones, such as metatarsal bones. Embodiments of the present invention relate to treatment of bone deformation disorders such as metatarsal bones and hallux valgus or repair of bone fractures such as Lisfranc.

Deformity of skeletal bones can affect posture, locomotion and the quality of life to of active individuals. Such deformity can be caused by traumatic injury or a creeping deformity.

Hallux valgus deformity is the most common forefoot disorder, with an estimated age related prevalence of 10 to 35%. Hallux valgus is characterized by outward deviation of the first metatarsal bone which leads to valgus deformity of the big toe (phalange). This deviation changes the biomechanics of the foot and may cause subluxation of the first metatarsophalangeal joint (MTP joint). Hallux Valgus is generally accompanied by bony eminence at the MTP joint area which is also referred to as a bunion. In severe cases, the great toe may even overlap the second toe. Non-operative treatment may alleviate symptoms but does not correct the deformity of the big toe. Surgical correction of hallux valgus (HV) is typically indicated when patient suffers from painful progressive deformity, and inhibition of activity or lifestyle. Surgical treatments for hallux valgus include corrective osteotomy in which the metatarsal bone of the great toe (First Metatarsal) is cut and repositioned reducing the IMA back to normal, resection arthroplasty in which a bone wedge is removed from the first MTP joint to reposition the great toe, or arthrodesis in which the first MTP joint is ossified in order to fixate the great toe in a correct position. The corrective osteotomy of the first metatarsal is followed by a long recovery which limits weight bearing activity and in many cases is accompanied by pain and discomfort.

In recent years, a number of minimally invasive approaches have been devised for correcting hallux valgus deformities. These approaches interconnect metatarsal bones under tension to restore the natural position of the bone and the great toe and maintain a normal IMA.

For example, U.S. Patent Application Publication 2010/0152752 and U.S. Pat. No. 7,875,058 describe an approach for bunion repair using a K-wire for passing a suture through the first and second metatarsal bones and correcting the inter-metatarsal angle deformity. An example of such a device, the Mini TightRope, is commercially available from Arthrex, Inc. (Naples, Fla.).

PCT International Publication WO 2009/018527 describes a fixation and alignment system for use in orthopedic surgery for the correction of bone deformities. The system is used to anchor two or more sections of bone or other body parts and to align one section relative to another and can be used in hallux valgus repair.

US 2010/0076504 describes a press-fit fastener body and coupler which is used in conjunction with a suture anchor and offers temporary or permanent fixation, restoring carpal alignment and normal range of motion.

PCT International Publication WO 2010/093696 describes an implantable tensioning device which includes a first anchor, a dynamic tension component coupled to the first anchor, and a second anchor coupled to the dynamic tension component. The first anchor is configured to be attachable to a first metatarsal bone and the second anchor is configured to be attachable to a second metatarsal bone. The dynamic tension component (elastic element or spring) has a tensioned state and an un-tensioned state. The tensioned state includes the component urging the first and second anchors toward each other.

WO/2012/029008 to the present inventor describes implantable device which includes two bone anchors interconnected by a cord and a shock absorber disposed in one of the bone anchors. The shock absorber includes a spring, which is configured to deform in response to a force exerted on the cord.

While the above described minimally invasive solutions can be used to restore alignment to metatarsal bones, there remains a need for a bone repositioning system which can be used to correct bone deformities such as hallux valgus.

### SUMMARY OF THE INVENTION

According to one aspect of the present invention there is provided a device for fixation of bone tissue comprising: (a) a first anchor positionable within a first bone region; (b) a second anchor positionable within a second bone region; and (c) a wire interconnecting the first anchor and the second anchor; wherein the wire is attached to the first anchor via an elastically deformable element having a force constant (K) of 20-80 N/mm along a longitudinal axis of the first anchor.

According to further features in preferred embodiments of the invention described below, the elastically deformable element is positioned within the first anchor.

According to still further features in the described preferred embodiment the elastically deformable element includes elastically deflectable projections.

According to still further features in the described preferred embodiment the projections elastically deflect as the elastically deformable element is advanced within to the first anchor.

According to still further features in the described preferred embodiment the deformable element is substantially tube shaped and the projections are longitudinal.

According to still further features in the described preferred embodiment the deformable element is substantially disc shaped and the projections are circumferential.

According to still further features in the described preferred embodiment the deformable element is tube shaped and includes vertical slits or cutouts.

According to still further features in the described preferred embodiment the deformable element is composed of an alloy.

According to still further features in the described preferred embodiment the alloy is a cobalt chrome alloy.

According to still further features in the described preferred embodiment the first anchor is an externally threaded hollow tube.

According to still further features in the described preferred embodiment a first end of the hollow tube includes an external flange and optionally a washer.

According to still further features in the described preferred embodiment a second end of the hollow tube includes an internal bevel.

According to still further features in the described preferred embodiment the first and the second anchors are sized and configured for placement within adjacent bones.

According to still further features in the described preferred embodiment the device is configured for interconnecting adjacent metatarsal bones.

According to still further features in the described preferred embodiment the wire has deformed ends.

According to another aspect of the present invention there is provided a device for fixation of bone tissue comprising: (a) a first anchor positionable within a first bone region; (b) a second anchor positionable within a second bone region; and (c) a wire interconnecting the first anchor and the second anchor, the wire having a deformed end.

According to yet another aspect of the present invention there is provided a device for tensioning a wire anchored to a bone, the device comprising a housing having a mechanism for engaging and tensioning the wire and a tension gauge for determining the force of tension.

According to still further features in the described preferred embodiment the to housing further comprises a proximal portion (close to the bone) for abutting bone tissue or an anchor disposed therein.

According to still further features in the described preferred embodiment the proximal portion includes a guide frame for positioning a wire deforming and/or cutting device against the wire engaged by the mechanism.

According to yet another aspect of the present invention there is provided a device for anchoring comprising an element having elastically deflectable projections positioned within an anchor having a lumen configured so as to deflect the fingerlike projections when the element is advanced within the lumen.

According to still another aspect of the present invention there is provided a method of interconnecting a first bone region to a second bone region, the method comprising: (a) positioning a wire between the first bone region and the second bone region; (b) delivering a first anchor over the wire and into the first bone region; (c) delivering a second anchor over the wire and into the second bone region; (d) deforming (preferably flattening) one or more ends of the wire to thereby trap the ends of the wire against the first anchor and the second anchor.

According to still further features in the described preferred embodiment (a) is effected by drilling holes through the first bone region and the second bone region using a cannulated drill bit carrying the wire within a lumen thereof.

According to still further features in the described preferred embodiment the first anchor includes an elastically deformable element and further wherein one deformed end of the wire is trapped against the elastically deformable element.

According to still further features in the described preferred embodiment the method further comprises a step of pulling the bones towards each other by tensioning the wire prior to (d).

According to still further features in the described preferred embodiment tensioning is effected via a device including a mechanism for engaging and tensioning the wire and a tension gauge for determining a tension between the bones.

According to still further features in the described preferred embodiment the wire is tensioned to a force of 20-80 N.

According to still further features in the described preferred embodiment the to elastically deformable element has a force constant (K) of 20-80 N/mm along a longitudinal axis of the first anchor.

According to still further features in the described preferred embodiment the first bone region is in a metatarsal and the second bone region is in an adjacent metatarsal.

According to still further features in the described preferred embodiment (a) is effected by drilling a straight hole through the metatarsal and the adjacent metatarsal using a cannulated drill bit carrying the wire within a lumen thereof.

The present invention successfully addresses the shortcomings of the presently known configurations by providing a bone anchoring system which can be used to interconnect adjacent bones for the purpose of treating bone fractures and skeletal deformities such as hallux valgus.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-C are isometric views of the implant device of the present invention in assembled (FIGS. 1A, C) and dissembled (FIG. 1B) states.

FIG. 1D illustrates a cross sectional view of the device of the present invention.

FIGS. 1E-F are cross sectional views of the anchor components of the present invention.

FIG. 1G is a cross sectional view of the present device implanted in adjacent bones.

FIGS. 2A-C illustrate a first embodiment of the elastic deformable element of the present device showing the element in isometric (FIG. 2A) and cross sectional (FIGS. 2B-C) views, with the elastically deformable element in normal (FIG. 2B) and deformed (FIG. 2C) states.

FIGS. 3A-C illustrate a second embodiment of the elastic deformable element of the present device showing the element in isometric (FIG. 3A) and cross sectional (FIGS. 3B-C) views, with the elastically deformable element in normal (FIG. 3B) and deformed (FIG. 3C) states.

FIGS. 4A-C are isometric views of a third embodiment of the elastic deformable element of the present device showing the element alone (FIG. 4A), when positioned against the anchor body (FIG. 4B) and deformed by a washer (FIG. 4C).

FIGS. 5A-D illustrate a fourth embodiment of the elastic deformable element of the present device showing the element in isometric view (FIG. 5A) and cross sectional views (FIGS. 5B-D). FIGS. 5B-C illustrate the deformable element in normal and deformed states (respectively), while FIG. 5D is a magnified view of a portion of the deformable element.

FIGS. 6A-M illustrate an intermetatarsal angle reduction procedure utilizing the present device.

FIG. 6N illustrates tibia fracture bone repair using the present device.

FIG. 7A-D illustrate embodiments of a small diameter cannulated drills which can be used with the present device in a bone repair procedure.

FIGS. 8A-Q illustrate a device for tensioning and deforming (e.g. flattening) a wire constructed in accordance with the teachings of the present invention. FIGS. 8A-B illustrate the device in isometric and cross sectional views (respectively). FIG. 8C is a magnified view of the tensioning device head; the wire deforming mechanism is to illustrated in FIG. 8D. The tensioning device and bone-implanted device are shown in FIG. 8E. Tensioned and non-tensioned states of the tensioning device are shown in FIGS. 8F-G (respectively). FIG. 8G illustrates the head of the tensioning device when interfaced with an implant anchor. FIG. 8I is a cross sectional of the head and the anchor. FIG. 8J-K illustrate the tensioning device when positioned at an angle to the anchor. FIGS. 8I, K illustrate the device when used along with an anchor having a conical head or a flat top head (respectively). FIGS. 8L-N illustrate the deforming mechanism and the wire prior to and following deformation. FIGS. 8O-P illustrate a device that can be used to actuate the deforming mechanism residing of the tensioning tool. FIG. 8Q illustrates another configuration of the device that can be used to deform a wire.

FIG. 9A-C are X-ray images of a human cadaver foot implanted with the present device under various wire tensioning forces.

FIG. 10A-B are X-ray images illustrating hallux valgus repair using the present device.

### DESCRIPTION OF THE PREFERRED EMBODIMENTS

The present invention is of a system and method which can be used to correct bone deformities such as those present in hallux valgus and to treat bone fractures. Specifically, the present invention can be used to realign the first metatarsal bone and restore alignment to the first MTP joint.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Minimally invasive approaches for correcting bone deformities such as those present in hallux valgus are well known in the art. While such approaches can be effective in aligning deformed bones, they are less effective at maintaining such an to alignment over extended periods of time due to breakage of anchors or tension cords, trauma to hard or soft tissue surrounding the anchoring site or mismatched dynamics between the tension applied to the bones and bone movement during activity.

While reducing the present invention to practice, the present inventor set out to correct the deficiencies of prior art approaches and provide a bone deformity correction system which is capable of:

(i) applying optimal tension between adjacent bones during periods of rest and activity;

(ii) minimizing trauma to bone and soft tissue during initial positioning and throughout treatment;

(iii) minimizing failure of tensioning members and anchors; (iv) enabling longitudinal elasticity with small displacement

(v) enabling controlled tensioning and force adjustment when interconnecting bones or bone fragments

Feature (i) above is of particular importance in hallux valgus repair. As is described in the Examples section which follows, the present inventor uncovered through experimentation that maintaining correct tension between the first and second metatarsal bones is pivotal to treatment and that prior art devices fail to provide the minimal tension forces required for treatment.

Prior art approaches, such as that described in WO/2012/029008 utilize elastic members such as springs to maintain a tension on the cord interconnecting the two bone anchors.

The strength of a spring is defined by the incremental force required to displace a spring by 1 mm (termed the “force constant” or K). The higher the K, the stronger the spring, i.e. the more force will be required in order to displace it a certain distance. In a conventional metal spring the force constant magnitude is a function of number of parameters such as the metal wire tensile strength, the thickness of the spring wire, the diameter of the spring, the spring length, the number of curves (coils) etc.

Based on the dimensions of the bone anchors of WO/2012/029008, a spring fabricated from the highest tensile strength biocompatible alloy (e.g. Cobalt Chrome—tensile strength >2000 MPa) with an outer diameter of 3.0 mm, a wire thickness of 0.50 mm, a length of 10 mm and about 10 coils would have a K of about 7-8 N/mm as is calculated using, for example, Advanced Spring Design software Ver. 7.0 developed by Universal Technical Systems (UTS) and Spring Manufacturers Institute (SMI). Under forces of about 30 N such a spring will contract about 4.5 mm. This force completely compresses the spring and as such, it will no longer respond to additional compressive forces.

As is described in the Examples section which follows, such a force would be substantially less than that required for maintaining longitudinal elasticity throughout natural bone movements.

In order to provide the optimal tensioning force and necessary longitudinal elasticity, the present inventor designed an elastic deformable element with small dimensions that can withstand high forces and provide a K of 20-80 N/mm. This high K provides longitudinal elasticity under forces between bones of 20-80 N.

Thus according to one aspect of the present invention there is provided a device for fixation of bone tissue.

The present device can be used for fixation of bone tissue of a single bone (e.g. for bone fracture repair), or for inter-fixation of two adjacent bones as is the case with hallux valgus deformity repair. The present device can be implanted in any bone tissue, including, but not limited to bone tissue of digits (e.g. metatarsals carpals, metacarpals, phalanges), vertebral bone tissue, long bones (e.g. femur, tibia, fibula, humerus, radius, ulna) forefoot and midfoot joint bones (e.g. for fracture repair) shoulder bones such as acromioclavicular joint (e.g. for fracture repair).

The device of the present invention includes a first anchor positionable within a first bone region, a second anchor positionable within a second bone region and a wire interconnecting the first and second anchors.

The wire is attached to the first anchor via an elastically deformable element having a force constant (K) of 20-80 N/mm along a longitudinal axis of the first anchor.

As is mentioned hereinabove and further described below, a deformable element having such a force constant is neither described not suggested in the prior art and provides advantages in hallux valgus deformity repair.

The first and second bone anchors of the present device are configured as substantially cylindrical hollow bodies composed of an implantable biocompatible metal or alloy such as cobalt chrome or stainless steel such as 316LVM, Titanium or biodegradable material such as magnesium or biocompatible plastic such as PEEK or an amorphous thermoplastic polyetherimide (PEI) resin such as ULTEM™.

The use of cobalt chrome in the present device (anchors and wire) is presently preferred. This alloy is particularly advantageous for use in anchors and wire since it is approved for long term implantation, it has a very high tensile strength, it can be deformed (e.g. flattened, to trap wire ends against anchors), it is highly elastic (a requirement for element 18 described hereinbelow), has low flexural rigidity (when drawn as an annealed or thermal stress released wire 16 described hereinbelow) and can be used for all device elements, thus traversing the problem of galvanic corrosion.

Depending on intended use and bone location, the anchors can be, for example, anywhere from 1.0 mm to 6.0 mm in diameter and 5.0 mm to 30.0 mm in length. Wire diameter can vary for example from 0.2-0.8 mm Specific dimensions are provided herein below with respect to the hallux valgus repair configuration of the present device.

Lumens extend the length of the first and second anchors and may vary internally. The wire is positioned through these lumens and secured to the second anchor body and to the elastic deformable element of the first anchor in the manner described below. The lumen can include portions of different diameters to suit the wire diameter and to accommodate the deformable element (described below). The lumen can be tubular or conical or any other shape suitable for accommodating the deformable element and attached wire.

The anchors are positioned within predrilled holes in bone tissue and preferably include an external thread for fixation to the bone tissue. At least one, preferably both anchors include a flange for abutting bone tissue in the direction of wire tension. This ensures that the anchor or anchors do not advance into the bone over time. The flange can include grooves/slots for engaging a screw driver head and holes to facilitate blood flow and enhance bone growth. The cross sectional shape of the flange can be flat, slightly rounded, conical or centrally extruded. The flange can be substituted or augmented by a washer.

The elastically deformable element of the present device can be attached to the to first bone anchor using one of several approaches. For example, the elastically deformable element can be attached to an end of the anchor body or it can reside within the lumen of the anchor body. In any case, the elastically deformable element elastically tensions the wire in along the longitudinal axis of the first anchor body.

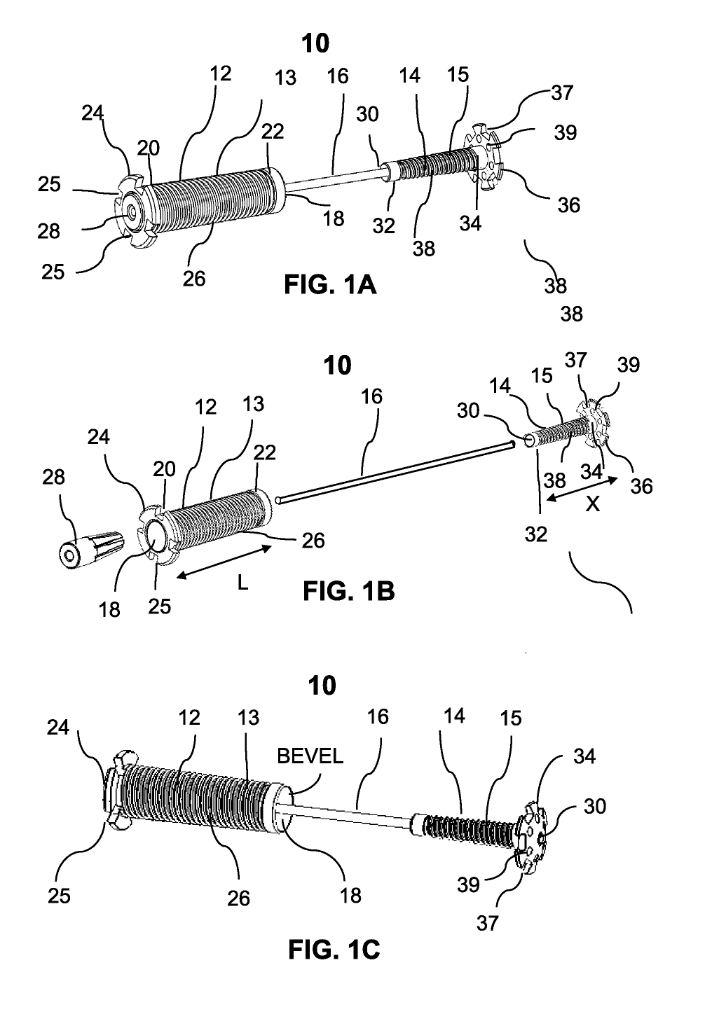

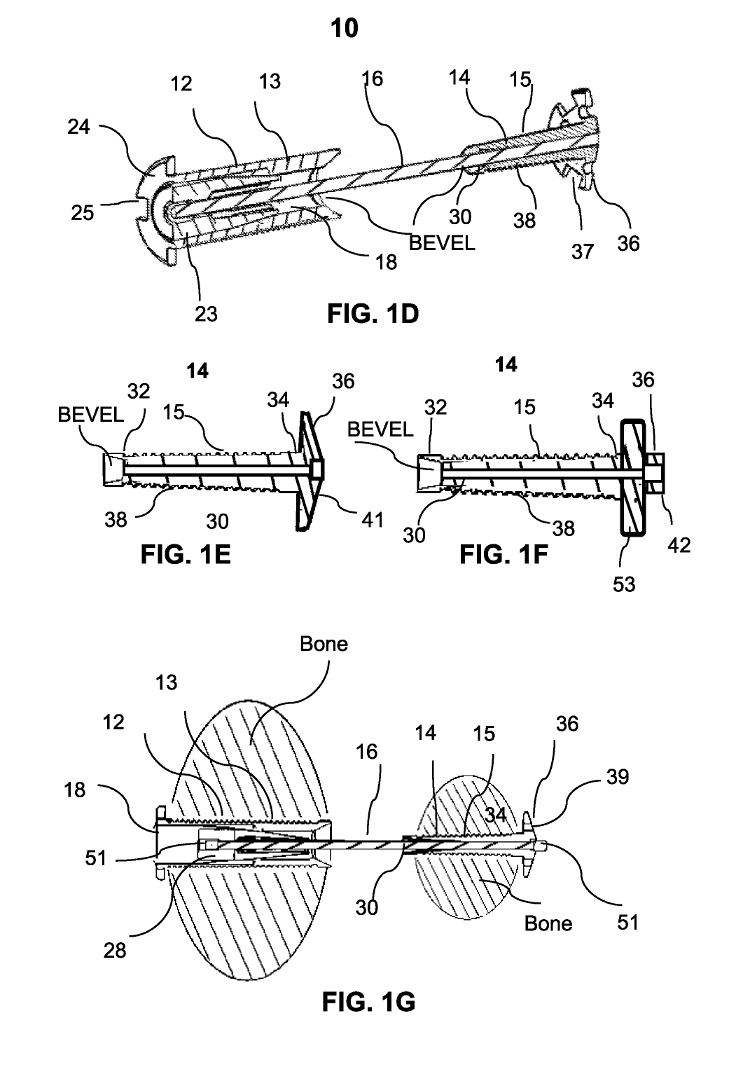

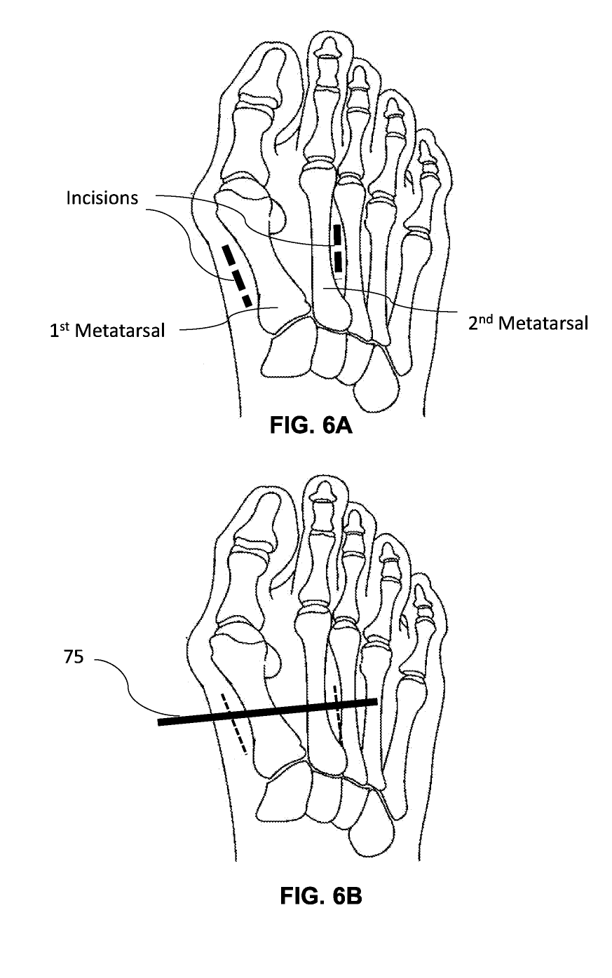

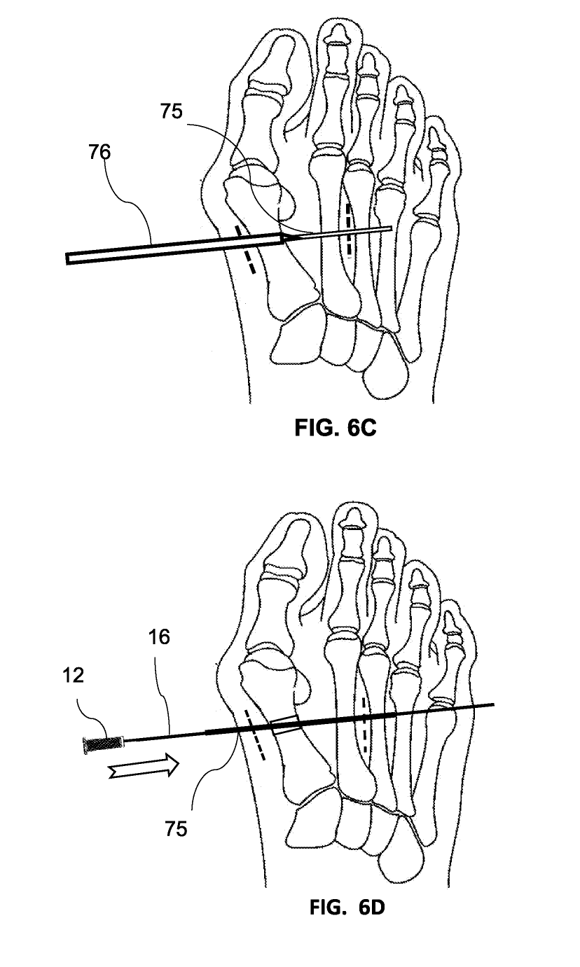

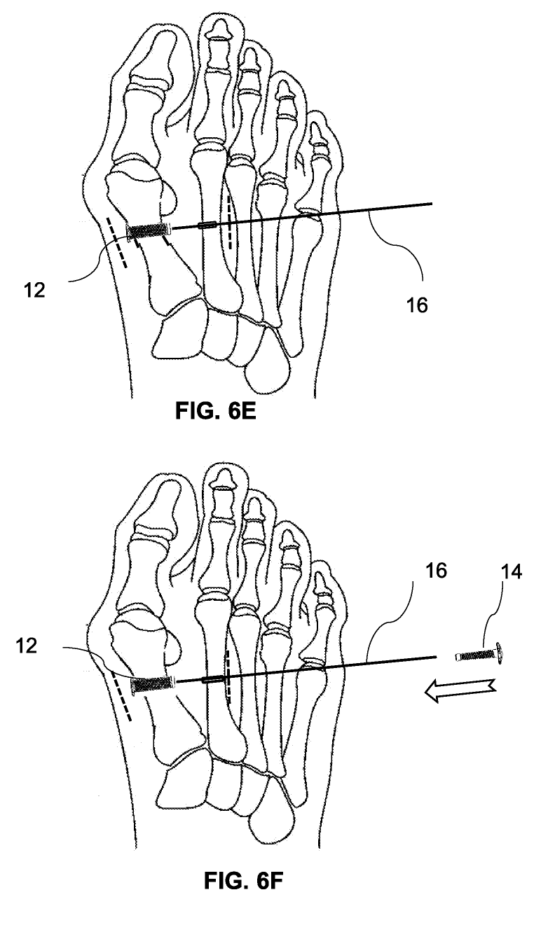

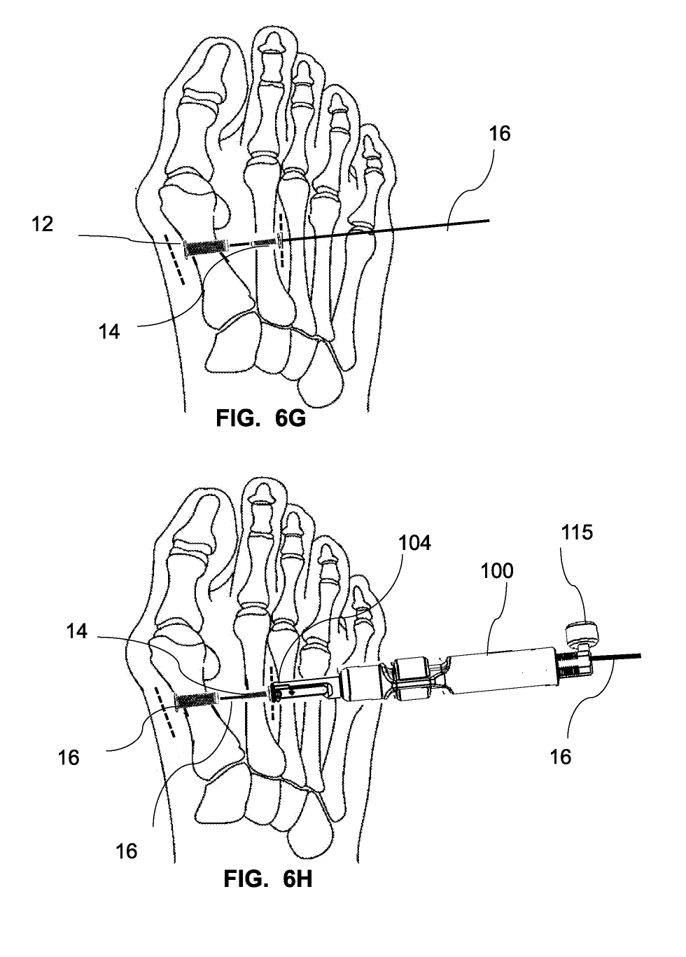

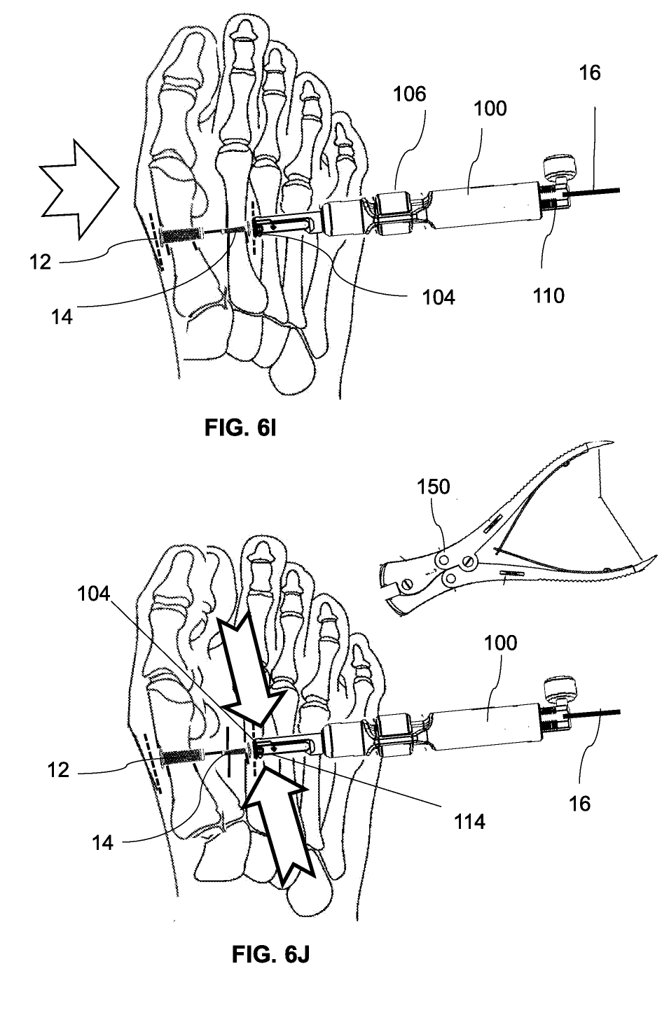

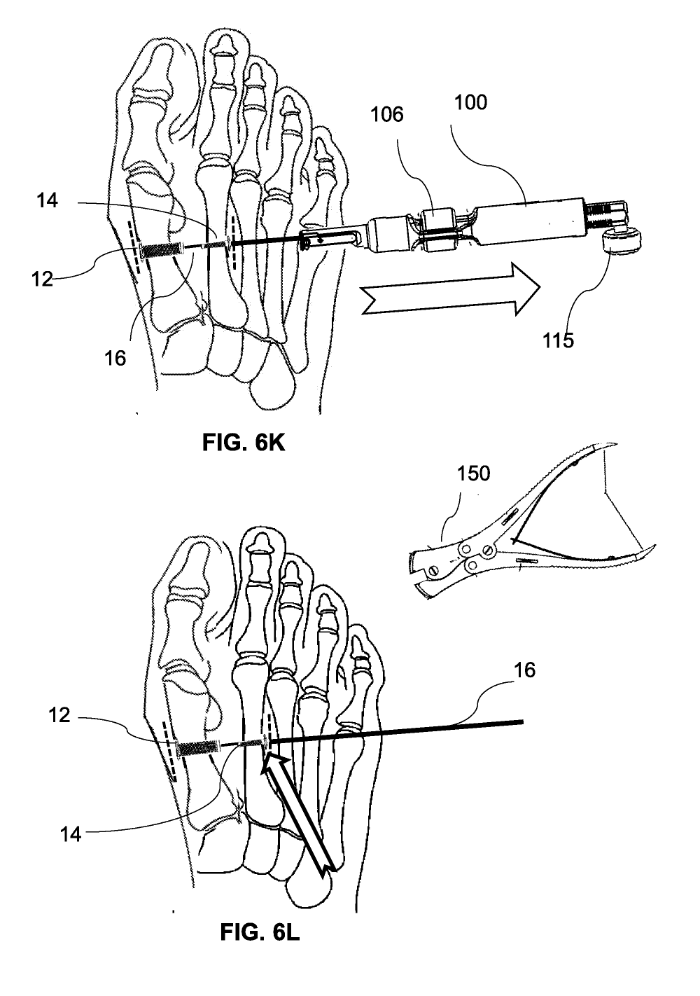

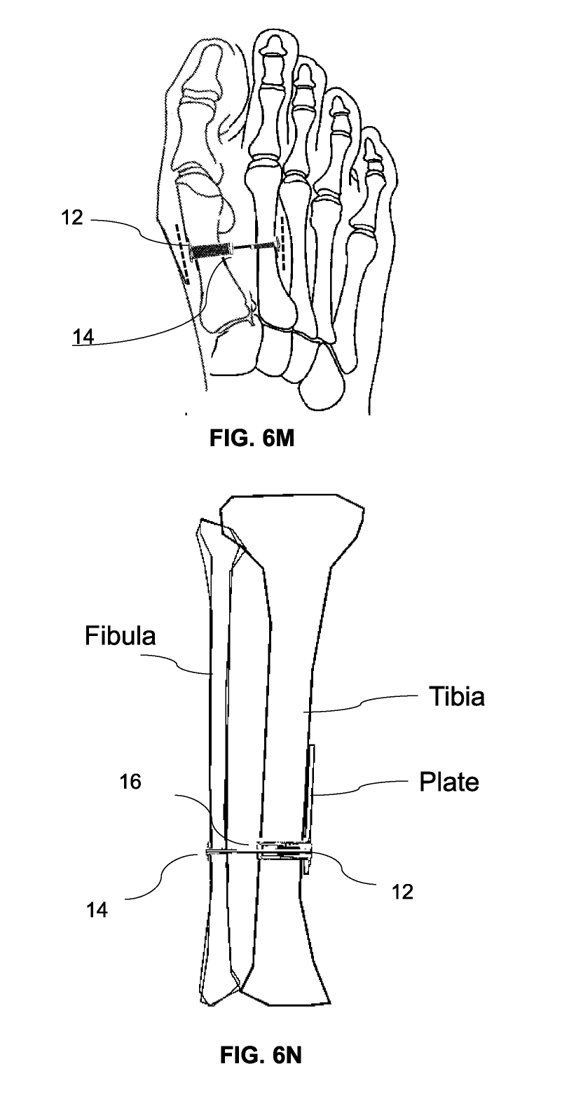

Referring now to the drawings, FIGS. 1-5d illustrate embodiments of the present device configured for use in hallux valgus bone deformity repair while FIGS. 6a-m illustrate the steps of using this embodiment of the present device in hallux valgus repair. It will be appreciated that the present device can also be configured for repair of bone fractures (as is shown in FIG. 6n) or other bone deformities by reconfiguring the anchors, elastically deformable element and/or wire for such purposes.

FIGS. 1a-g illustrate the present hallux valgus deformity repair device which is referred to herein as device 10.

Device 10 includes a first anchor 12 and a second anchor 14 which are inter-connectable via a wire 16. First anchor 12 (also referred to herein as proximal anchor 12 or anchor 12) includes an anchor body 13 (also referred to herein as body 13) which is substantially cylindrical. Body 13 includes a lumen 18 running along a length (L) thereof preferably extending from a proximal end 20 to a distal end 22 of body 13. Lumen 18 can have uniform or varying diameter and cross sectional shape. Lumen 18 is preferably cylindrical and/or conical in shape or any other shape suitable for accepting elastically deformable element 28 (further described below) and/or wire 16. As is shown in FIG. 1d, the diameter of lumen 18 can vary along L and may include a first wide portion contiguous with a second narrower portion contiguous with third wide portion.

Proximal end 20 of body 13 includes a flange 24 and/or a washer (not shown) for abutting bone tissue and preventing migration of body 13 into the bone when anchor 12 is forced in a distal direction (in the direction of anchor 14) under tension of wire 16. Flange can include detents 25 for enabling threading of body 13 into the bone and openings for facilitating bone growth and blood circulation.

Anchor body 13 can include external tissue anchoring elements 26 (e.g. threads) along at least a portion of its length. Such elements 26 help stabilize and integrate anchor body 13 into the bone tissue.

Anchor body 13 can be fabricated from a biocompatible long term implantable metal or alloy such as cobalt chrome, stainless steel, titanium, biodegradable material such as Magnesium, biocompatible plastic material such as PEEK or ULTEM via molding, forging, machining or any combination thereof. When utilized in hallux valgus repair, typical dimensions for body 13 are 10-18 mm length and 3-5 mm OD with an average lumen 18 diameter of 2-3 mm. The diameter of wire 16 can be 0.2-0.8 mm. The diameter of flange 24 and/or the washer can be 4-6 mm.

Anchor 12 further includes an elastically deformable element 28 which is positionable within lumen 18 or against distal end 22 of anchor body 13. Elastically deformable element 28 is attachable to a proximal end of wire 16 and serves to elastically compensate for changes in a distance between anchors 12 and 14 when they are anchored to bones (e.g. metatarsal bones) and interconnected via wire 16. Deformable element 28 can be attached to wire 16 via, for example, laser welding or by deforming wire 16 ends as is described hereinbelow. The elastic nature of elastically deformable element 28 ensures that a tension on wire 16 remains relatively unchanged throughout such changes in distance, thus maintaining a substantially uniform repositioning force on the first metatarsal. As elastically deformable element 28 is pulled by wire 16 onto lumen 18 of body 13, it elastically deforms to increase tension on wire 16 and vice versa.

Elastically deformable element 28 is configured to provide a force constant (K) of 20-80 N/mm along a longitudinal axis of anchor 12. As is mentioned hereinabove, such a force constant is substantially larger than that providable by prior art devices having elastic wire tensioning mechanisms.

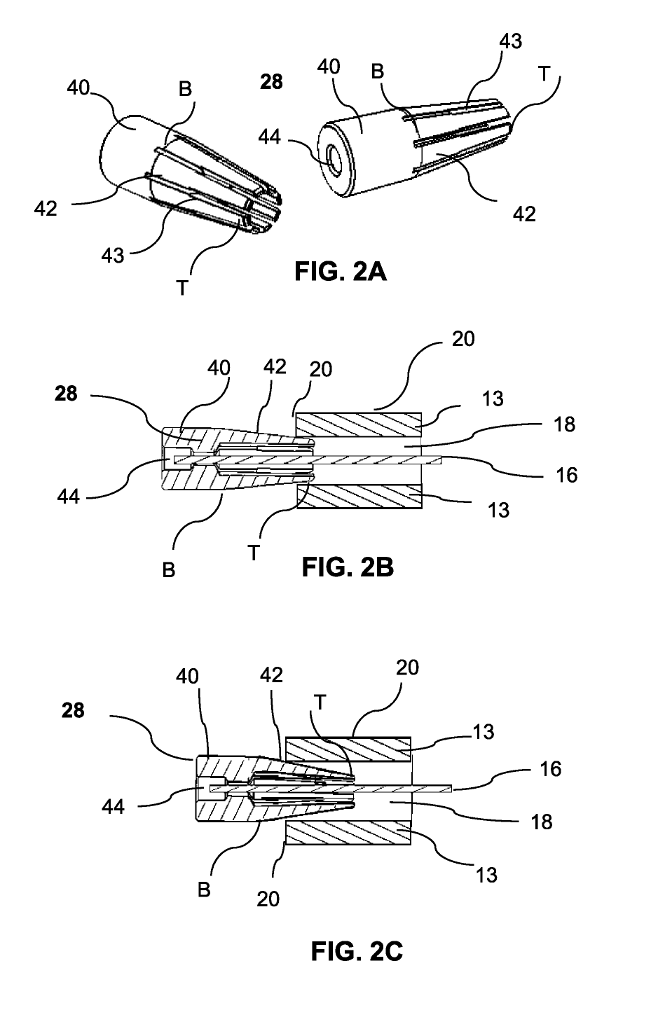

Several embodiments of elastically deformable element 28 can be used to provide such a force constant when used in conjunction with anchor body 13. A detailed description of several elastically deformable element 28 embodiments is provided below with reference to FIGS. 2a-5d.

Device 10 further includes anchor 14 (also referred to herein as distal anchor 14, or anchor 14), which in the case of hallux valgus deformity correction is positioned in the second metatarsal directly opposing proximal anchor 12.

Anchor 14 includes an anchor body 15 (also referred to herein as body 15) which is substantially cylindrical. Body 15 includes a lumen 30 running along a length (X) to thereof and preferably extending from a proximal end 32 to a distal end 34 of body 15. Lumen 30 can have a similar narrowing as that of lumen 18 described above. Lumen can be cylindrical or conical, a combination of both or any other shape suitable for running of wire 16 there through.

Body 15 includes a flange 36 for abutting bone tissue and preventing migration of body 15 into the bone when anchor 14 is forced in a proximal direction (towards anchor 12) under tension of wire 16. Flange 36 can be substituted or augmented by a washer 53.

Flange 36 (and washer 53) can include detents 37 or holes 39 for enabling threading of body 13 into the bone hole and can have additional holes for facilitating bone growth and blood circulation. When flange 36 is used in combination with washer 53, it will be of a smaller diameter and will include extrusion 41 positioned outside washer 53.

Anchor body 15 can be cylindrical or conical in shape. Anchor body 15 can include external tissue anchoring elements 38 (e.g. threads) along at least a portion of its length. Such elements 38 help stabilize and integrate anchor body 15 into the bone tissue.

Anchor body 15 can be fabricated from a metal or alloy such as cobalt chrome, stainless steel, or titanium, magnesium or biocompatible plastic material such as PEEK or ULTEM via molding, forging, machining or any combination thereof. For example, when utilized in hallux valgus repair, typical dimensions for body 15 are 9 mm length and 1.8 mm OD with an average lumen 30 diameter of 1.2 mm. The diameter of flange 36 or washer 53 can be 5.0 mm.

Wire 16 is attached to anchor 12 via elastically deformable element 28 and to body 15 of anchor 14. Wire 16 is attached to body 15 by deforming wire 16 end as is described hereinbelow. Wire 16 can be fabricated from a metal, alloy (preferably cobalt chrome), from a polymer such as Nylon. Wire 16 can be a single filament wire or a braided wire and can be circular, square or rectangular (flat) in cross section.

As is mentioned hereinabove, anchor 12 of device 10 includes an elastically deformable element 28 for maintaining tension of wire 16 interconnecting anchors 12 and 14.

FIGS. 2a-5d illustrate several embodiments of elastically deformable element 28 and of lumen 18 of anchor 12. As is further described below, lumen 18 is specifically shaped for use with each specific embodiment of elastically deformable element 28 in order to provide the elastic deformation of elastically deformable element 28 necessary for regulate tension on wire 16.

FIG. 2a illustrates a first embodiment of elastically deformable element 28. FIG. 2b illustrates elastically deformable element 28 positioned into body 13 of anchor 12, while FIG. 2c illustrates elastically deformable element 28 pulled into body 13 (as is the case under tension of wire 16) and deformed.

Elastically deformable element 28 includes a cylindrical body 40 and several projections 42 extending along body 40 and forming a slightly conical shape (projections 42 are slightly angled inward). Projections 42 are separated via slits 51 for accommodating deformation of projections 42. Cylindrical body 40 can have a typical diameter of 2-3 mm at base (B) and 1.5-2 mm at tip end (T). Projections 42 can be rectangular or trapezoidal in shape with a typical length of 2-5 mm, slit 43 can have a width of 0.1-0.3 mm at the base (B) and a width of 0.2-0.4 mm at the tip end (T). The number of projections 42 can range from 3 to 12. The thickness of projections 42 can be constant or variable from base to tip. Typical thickness at the base (B) can be 0.2-0.5 mm and typical thickness at the tip end (T) can be 0.1-0.5 mm. The K constant of deformable element 28 can vary depending on material type, length of projections 42, number of projections, slit size 43 and dimensions of projections 42 and projection 42 thickness. For example, an elastically deformable element 28 such as that shown in FIG. 2a with outer diameter (OD) at base of 2.8 mm and an OD of 1.9 mm at tip end, eight projections with a thickness at the base of 0.6 mm and 0.2 mm at the tip end and slits 43 having a width of 0.2 mm at the base and 0.3 mm at the tip end tensioned into a 2.0 mm anchor lumen has a K of about 33 N/mm and can move about 1.5 mm into the lumen of body 13.

Projections 42 can be formed by cutting (e.g. laser or CNC) body 40 or by molding elastically deformable element 28. Projections 42 are capable of elastically deforming radially inward when pushed into a cylindrical lumen 18 which is slightly narrower in diameter than the diameter of body 40 at the tip end (or mid-body) of projections 42. Thus, when a wire 16 is attached to elastically deformable element 28 (via laser welding, crimping or deforming) the end of the wire extending through and out to of elastically deformable element 28 thereby trapping it outside it against hole 44), and the wire is tensioned downward (pulling elastically deformable element 28 into the lumen 18 of body 13), projections 42 elastically deform radially inward and generate a counter force on the wire attached thereto. Such an elastic counter force increases as the tension on the wire increases since elastically deformable element 28 migrates further downward into the lumen of body 13 thereby increasing the deformation (and elastic response) of projections 42. As tension on the wire decreases, projections 42 rebound radially outward with movement (towards proximal end 20) of elastically deformable element 28 thereby maintaining tension on wire 16.

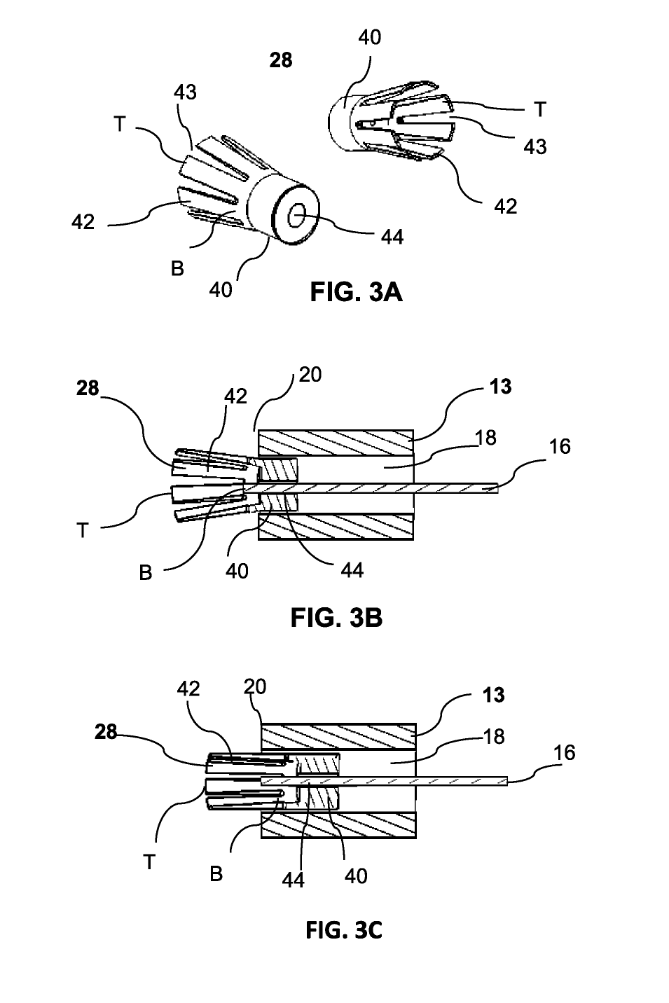

FIG. 3a illustrates a second embodiment of elastically deformable element 28. FIG. 3b illustrates elastically deformable element 28 slightly pushed into body 13 of anchor 12, while FIG. 3c illustrates elastically deformable element 28 pulled into body 13 (under tension of wire 16).

Elastically deformable element 28 of FIGS. 3a-c is similar in configuration to that shown in FIGS. 2a-c, however in this embodiment, projections 42 are slightly angled outward such that the diameter at the tip end (T) is larger than at the base (B) of projections 42. Diameter of body 40 of elastically deformable element 28 is slightly smaller than lumen 18 of body 13 and is inserted in a reverse orientation to that shown in FIGS. 2a-c with the base 40 inserted first. Thus, when a wire 16 is attached to elastically deformable element 28 and is tensioned downward (pulling elastically deformable element 28 into the lumen 18 of body 13), projections 42 elastically deform radially inward and generate a counter force on the wire attached thereto. Such an elastic counter force increases as the tension on the wire increases since elastically deformable element 28 migrates further downward into the lumen 18 of body 13 thereby increasing the deformation (and elastic response) of projections 42. As tension on the wire decreases, projections 42 rebound radially inward with movement (towards proximal end 20) of elastically deformable element 28 thereby maintaining tension on wire 16.

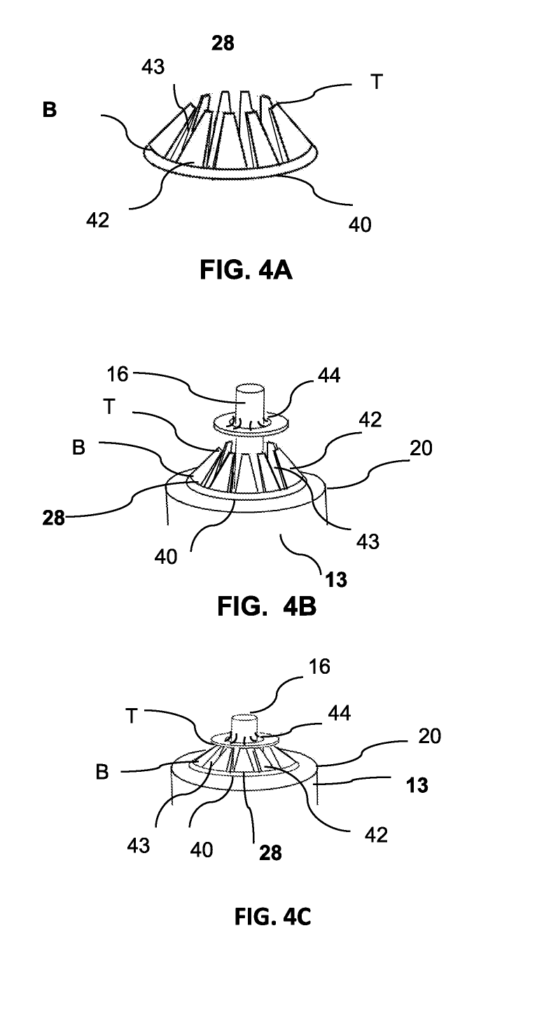

FIG. 4a illustrates a third embodiment of elastically deformable element 28, shown in isometric view. FIG. 4b illustrates elastically deformable element 28 positioned against body 13 with lumen 18 of anchor 12, while FIG. 4c illustrates elastically deformable element 28 pulled against body 13 (as is the case under tension of wire 16).

In this embodiment, elastically deformable element 28 includes a wide cylindrical base 40 and inward angled projections 42. Base maintains elastically deformable element 28 against an end surface 20 of body 13, and as wire 16 (along with disc 50) is pulled inward (FIG. 4c) disc 50 contacts the tips of projections 42 and deforms them 42 inward and down to create an elastic counter force. As tension on wire 16 decreases, projections 42 rebound upward, thereby maintaining tension on wire 16.

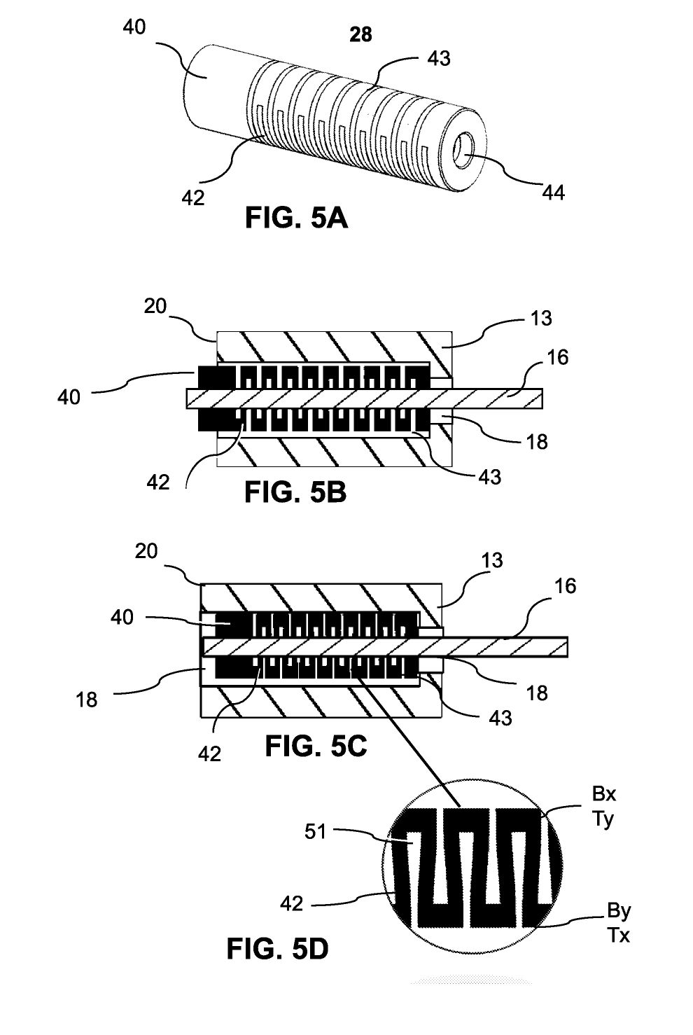

FIG. 5a illustrate a fourth embodiment of elastically deformable element 28 which is shaped as a cylinder 40 with lumen 44 and deformable alternating projections 42 and slits 43 (cut via CNC or laser) which are aligned vertically to the movement axis and are positioned in a mirrored direction and shifted such that tip end (T) is connected to base of an opposite projection.

FIG. 5b illustrates element 28 positioned in a lumen 18 of anchor 13 and being in a normal (non-compressed/deformed) state. Lumen 18 has smaller diameter towards distal end 24. When tension is applied to wire 16, projections 42 deform along the direction of movement as is shown in FIG. 5c. FIG. 5d is a magnified view of the deformation. Wire 16 can be threaded through a lumen 40 of elastically deformable element 28 and attached thereto as described herein.

An element 28 made of cobalt chrome with an outer diameter of 2.4 mm and inner diameter of 0.9 mm, 8 mm in length with alternating straight slits 0.18 mm in width and spaced apart by 0.7 mm has a K of 30 N/mm Such an element 28 can compress inward 1.5 mm at force of about 50 N.

As is mentioned hereinabove, device 10 of the present invention can be used in repair or fixation of any skeletal bone(s). One preferred use of device 10 is in correction of bone deformity in hallux valgus disorder.

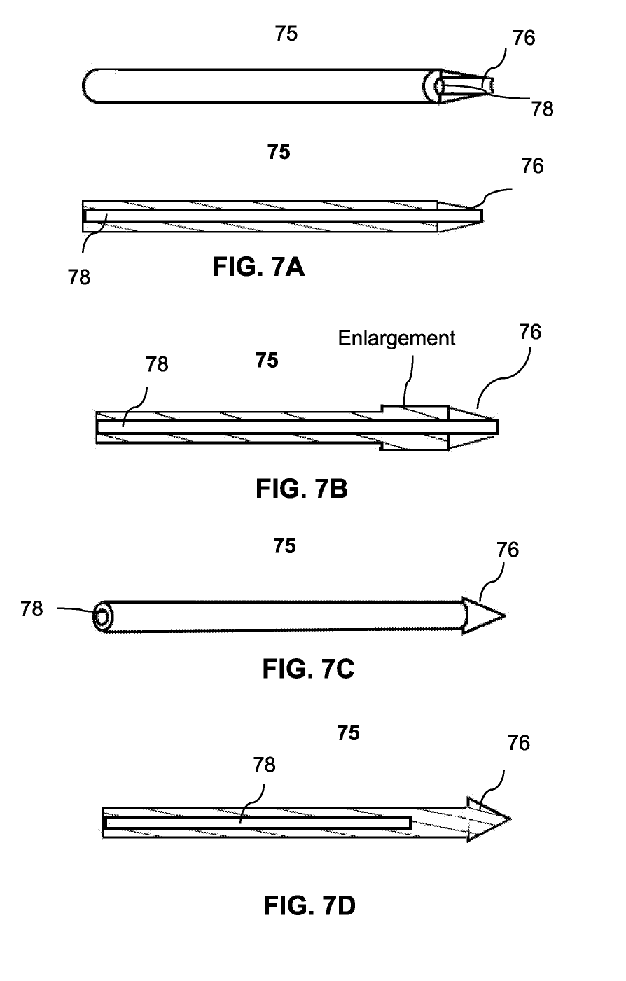

The following describes a hallux valgus deformity repair procedure using device 10 of the present invention. The procedure described hereinunder relates to the use of device 10 for first metatarsal realignment.
(i) A ˜2 cm skin incision is made at the lateral side of the second metatarsal. A similar incision is performed at the medial side of the first metatarsal (FIG. 6a).
(ii) A drill guide (not shown) may be positioned across the first and second metatarsals at about mid-shaft position and at about center bone height line. A small diameter hole of about 1.5 mm in diameter is drilled through the first and second metatarsal using a cannulated drill bit 75 (described in detail herein below with respect to FIGS. 7a-d). The drill guide is removed while the small diameter cannulated drill bit 75 is maintained in its position in the bones (FIG. 6b).
(iii) A 3.5 mm diameter hole is drilled through the first metatarsal using a cannulated drill bit positioned over the cannulated small diameter drill which serves as a guide (FIG. 6c). Drill bit 75 is maintained in its position between the bones.
(iv) Anchor 12 is attached to wire 16 and inserted into the lumen of the small diameter cannulated drill 75 (FIG. 6d).
(v) Drill bit 75 is then advanced outward (laterally) together with wire 16 and then removed. Anchor 12 is threaded into the hole of the first metatarsal bone (FIG. 6e) using a dedicated screw driver head that interfaces with flange 24.
(vi) Bone anchor 14 is advanced over wire 16 (FIG. 6O and threaded into the hole in the second metatarsal using a dedicated screw driver head. Both anchors 12 and 14 are tightly threaded into the bones until their flanges abut the bone surface (FIG. 6g).
(vii) A wire tensioning and flattening device 100 (described in details below), is advanced over wire 16 until wire comes out of the distal end of device 100. Device 100 head 104 is positioned over flange 36 (of anchor 14) and wire 16 is secured by closing and tightening knob 115 (FIG. 6h).
(viii) Device 100 is then used to tension the wire by rotating knob 106 (clockwise). Tensioning can be performed in a controlled manner observing the level of the tension force on force indicator 110. A combination of a predetermined tension force (e.g. 40 N) and optimal bone alignment (angle of 4-9°) as indicated visually and verified by imaging (FIG. 6i) is set. Device 100 is then used to secure the wire under tension at the appropriate position (FIG. 6j) by, for example, flattening the wire ends using instrument 150 positioned over lips 114.
(ix) Device 100 is then removed (FIG. 6k) and the remaining wire beyond the flattened region is cut and removed (FIG. 6l). The incision sites are then sutured closed (FIG. 6m).

FIGS. 7a-d illustrate several embodiments of small diameter cannulated drill bit 75. Drill bit 75 is fabricated from small diameter biocompatible stainless steel tube (e.g. 316L, 420). Tip 76 is beveled or pointed to enable drilling into the bone.

Various shapes of tip 76 are contemplated herein, with several embodiments shown in FIGS. 7a-d. Tip 76 may have a larger diameter than the shaft of drill bit 75 to enable pulling out in a smooth way drill bit 75 following drilling. Typical length of drill bit 75 can be 100 mm, while the length of tip 76 can be 4-8 mm and its outer diameter can be 1.5 mm. The OD of shaft of drill bit 75 can be 1.3 mm. The inner diameter (ID) of the lumen of drill bit 75 can be 0.5-0.7 mm FIGS. 7c-d depict a partially cannulated drill bit 75, where tip 76 is solid and the shaft is cannulated. Such a configuration enables to provide a tip 76 which is more efficient in penetrating bone.

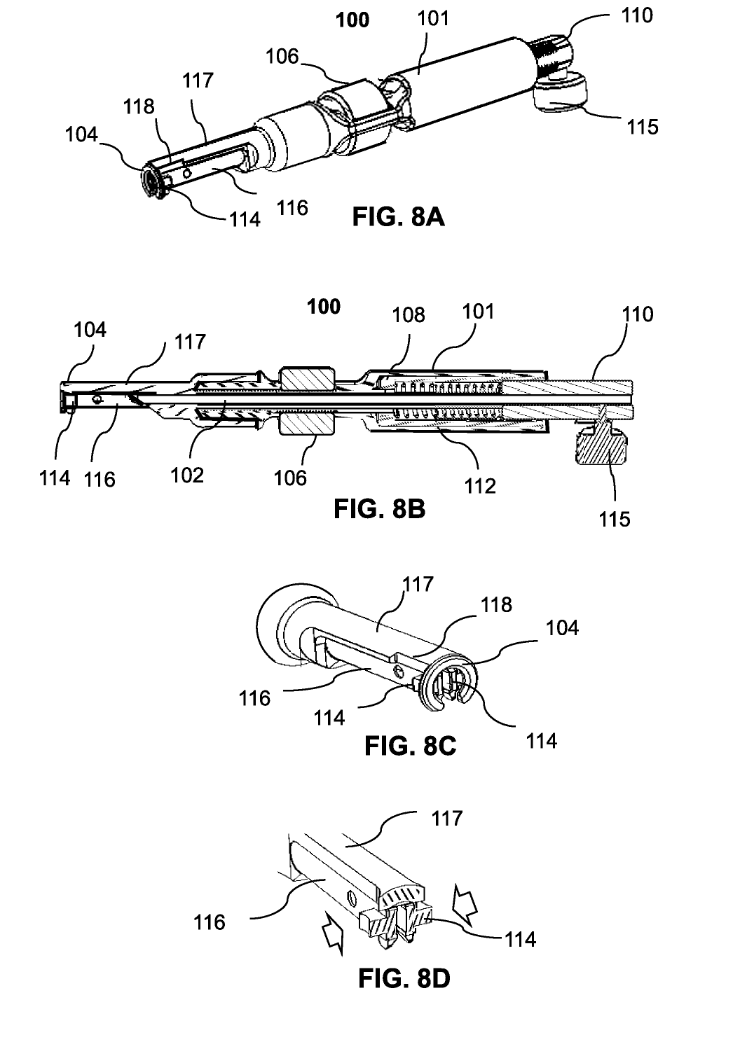

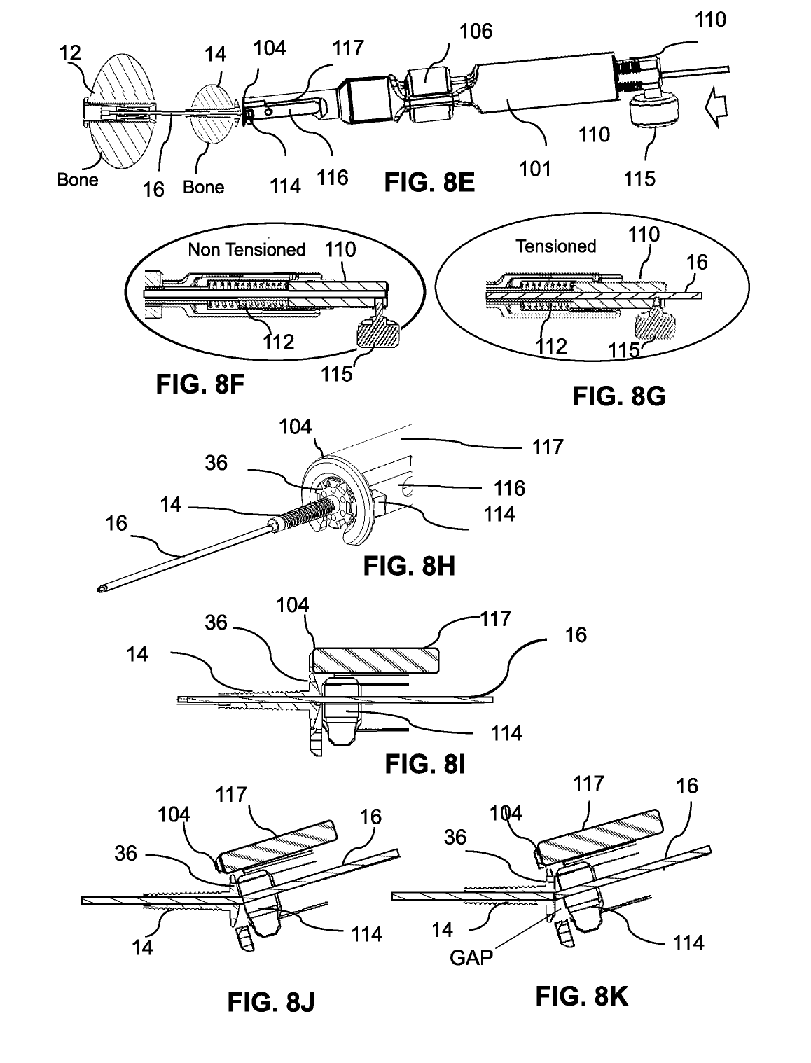

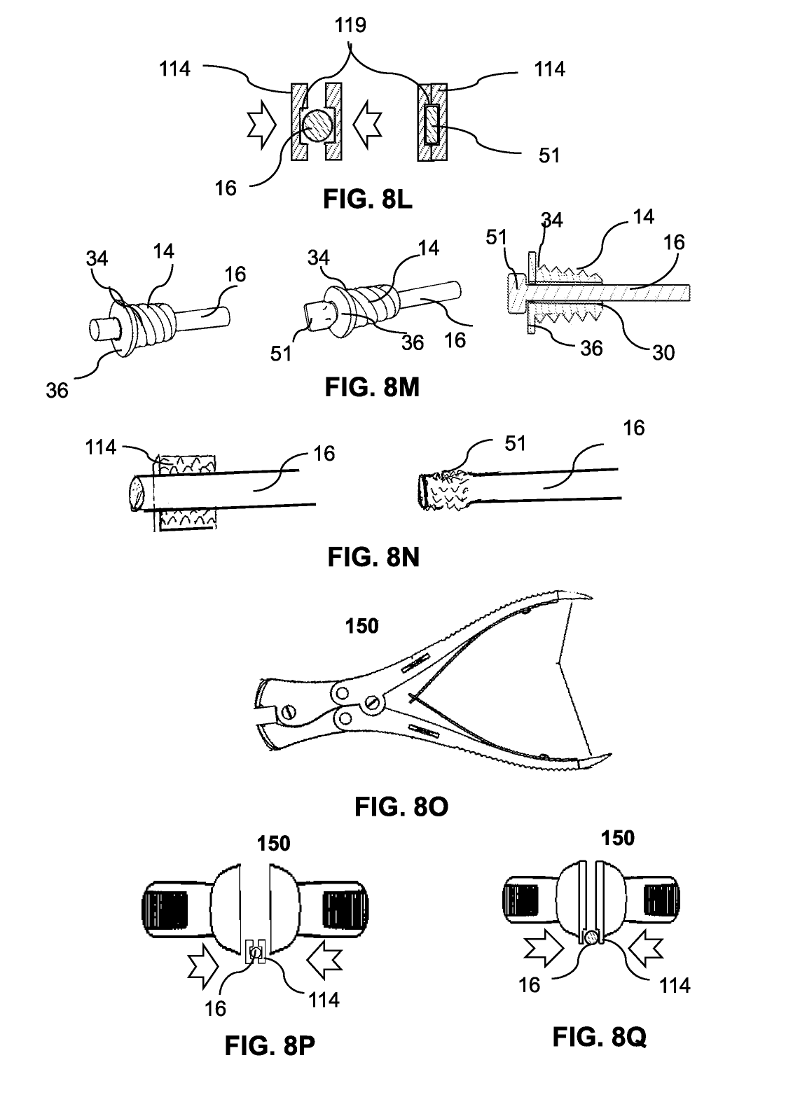

Tensioning device 100 is shown in FIGS. 8a-k. Device 100 has a housing 101 having longitudinal lumen 102 for accepting wire 16. Device 100 and its parts can be fabricated from a biocompatible metal, alloy or biocompatible polymer or combination of both. For the matter of example device 100 has the following general dimensions: 120 mm length and 14 mm in diameter.

Housing 101 includes a proximal part 117 and a distal ring-like element 104 which is positionable around anchor 14 head. A knob 106 which is rotatable over a threaded rod 108 which resides internally in house 101, wire 16 is attached to housing via screw knob 115. When rotating knob 106 in one direction (e.g. clockwise) rod 108 moves in a distal direction along the longitudinal axis of housing 101, rotating it in the opposite direction (counterclockwise) moves rod 106 in an opposite direction. When rod 108 is moved laterally (away from anchor 14) it pulls wire 16 and reduces the distance between anchors 12 and 14. As the tension force on wire 16 increases, spring 112 retracts and indicator 110 moves into housing 101. The inward movement of indicator 110 is proportional to the tension force applied to wire 16. Indicator 110 includes graduated marks which provide an indication of the tension on wire 16. Such a to mechanical tension indicator can be replaced via a load cell sensor, a pressure sensor or the like.

Proximal part 117 of device 100 includes a pair of ‘lips’ 114 for deforming wire 16 at the lateral end of anchor 14 to form flattened end 51. Lips 114 are fabricated from a hardened material such as hardened 402 stainless steel or cobalt chrome. Lips 114 (shown in details in FIGS. 8c-d) are residing on two elastic parallel plates 116 and are capable of parallel inward movement.

Proximal part 117 has a narrowed neck 118 that provides an accurate location for positioning a pressing device 150 (FIG. 8n). When device 150 (FIG. 8o) is positioned in neck 118 and pressed over lips 114 (FIG. 8p), wire 16 is deformed (FIG. 8I) so as to trap from moving into lumen 18 of anchor 14.

Lips 114 have an internal cavity 119 (FIG. 8I) that limits the amount of flattening and thus enable controlled, repeatable dimensional flattening of wire 16 at a pressing force which is above a predetermined minimum. Device 150 can have integral lips 114 and can be used to deform wire 16 in cases where tensioning is not required (FIG. 8q).

Deformed wire 16 can be, for example, rectangular, triangular or include protrusions such as shown in FIG. 8n. Shapes 51 will depend on the shape of cavity 119.

A cobalt chrome wire 16 having a diameter of 0.49 mm can be deformed (pressed) by lips 114 and a double action cutter-like device 150 to a substantially flat rectangular shape 51 of 0.38 mm in thickness and 0.65 mm in height. When pulled against a lumen 18 having a diameter of 0.50 mm the flattened wire can resist 200 N of force.

It is expected that during the life of this patent many relevant alloys will be developed and the scope of the term alloy is intended to include all such new technologies a priori.

As used herein the term “about” refers to ±10%.

Additional objects, advantages, and novel features of the present invention will become apparent to one ordinarily skilled in the art upon examination of the following to examples, which are not intended to be limiting.

### EXAMPLES

Reference is now made to the following examples, which together with the above descriptions, illustrate the invention in a non limiting fashion.

### Example 1

### Cadaver Study

A study was conducted in order to evaluate the transverse inter-metatarsal forces between first and second metatarsals after reduction of inter-metatarsal angle (IMA) in normal foot and in case of hallux valgus deformity.

Four fresh frozen cadaver feet (one with Hallux valgus) were used in the study. The device of the present invention was implanted in all four cadaver feet. The device was positioned between 1st and 2nd metatarsals at mid-shaft and the IMA was reduced using the dedicated wire tensioning device of the present invention. The tool includes a force indicator that shows the transverse load between the two metatarsals.

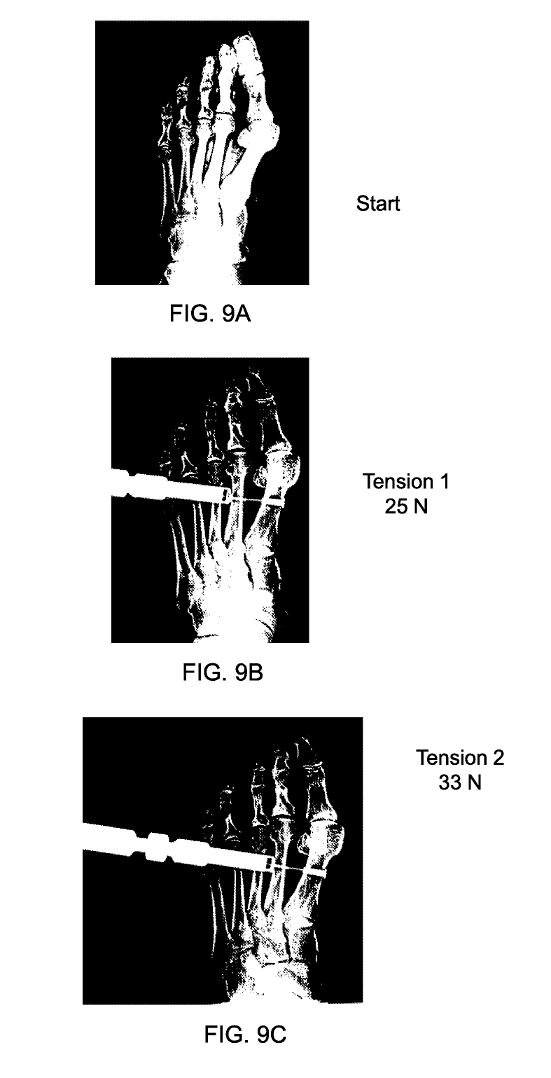

Each of the four feet was tensioned gradually reducing the IMA. Force was recorded and X-Rays were obtained (FIGS. 9a-c). Three cadaver feet were also loaded at 15° tilt under body weight and inter metatarsal force under load was recorded.

Results

Three of the cadaver feet exhibited a normal IMA (less than 10 Degrees) and one exhibited hallux valgus deformity (15 Degrees). Average weight of the 4 donors was 60.7 Kg (STD 14.5 Kg). Average initial IMA was 10.3 Deg. (STD 3.7 Deg), IMA was reduced by 4.7 Deg. (STD 1.9 Deg.). Average recorded Transverse Force was 28.5 N (STD 4.2 N), increase of transverse force at weight bearing 15° tilt was 6.3 N (STD 2.6 N).

Conclusions

Direct measurements of inter-metatarsal forces between 1st and 2nd metatarsals at mid-shaft indicated that a force of about 30 N is needed in order to reduce the IMA by about 5 degrees. The study also indicated that loading the foot at body weight increases the inter-metatarsal force by about 6 N.

### Example 2

### In Vivo Human Study

A clinical study was conducted in order to evaluate the efficacy and safety of the present device in human subjects. The feet of five female patients ages 22-67 having moderate Hallux Valgus were implanted with the present device in order to realign the first metatarsal to a normal position.

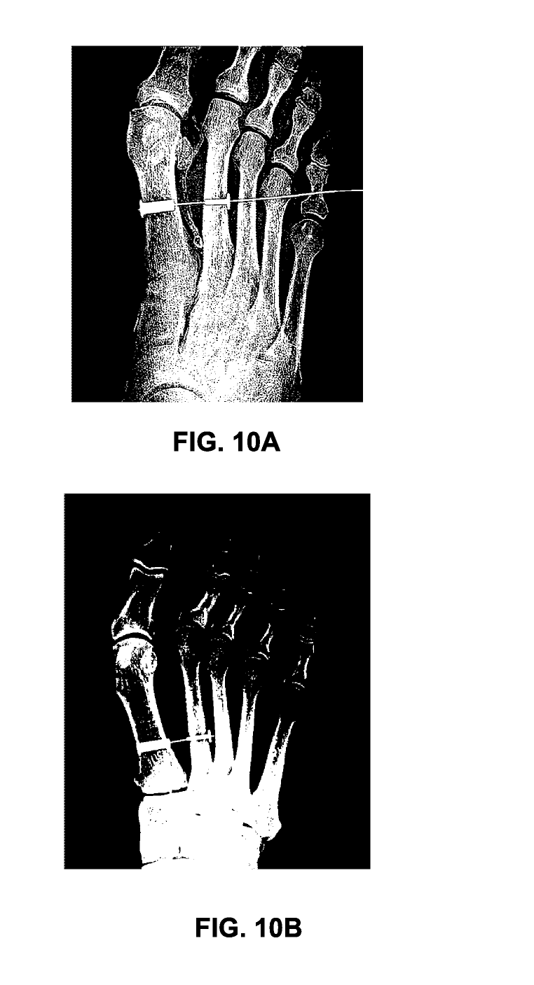

The device of the present invention was implanted as described herein and the force indicator of the tensioning device was used to measure the transverse load between the two metatarsals. The force was recorded and an X-Ray was taken (FIGS. 10a-b) without loading the foot.

Results

The average pre-op Inter Metatarsal Angle (IMA) was 14.60 (STD 0.80) and the average reduction was by 8 degree to a final 6.60 degrees (STD 0.630). The device was positioned at different distal distances from the cuneiform joint of the first metatarsal at an average distance of 35.4% (STD 5.3%) of the first metatarsal length measured at base of bone (Cuneiform joint). The average tensioning force was 35.4 N (STD 5.4 N). Tensioning force was assessed for different device positions. Assuming a linear moment, if all of the implanted devices were positioned distally at 40% (Measured from bone base) and 50% (mid shaft) of the first metatarsal length, the average tensioning force would have been 32 N (STD 8.2 N), 26 N (6.6 N) respectively.

Conclusions

Direct measurements of inter-metatarsal forces between the first and second metatarsals at about 35% of bone length indicated that a force of about 35 N is needed in order to reduce the IMA to normal values by about 8 degrees. If measured at center of bone these forces can be reduced to 26 N (STD 6.6 N).

It is appreciated that certain features of the invention, which are, for clarity, described in the context of separate embodiments, may also be provided in combination in a single embodiment. Conversely, various features of the invention, which are, for to brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A device for fixation of bone tissue comprising:
(a) a first anchor positionable within a first bone region;
(b) a second anchor positionable within a second bone region; and
(c) a wire interconnecting said first anchor and said second anchor;
wherein said wire is attached to said first anchor via an elastically deformable element having a force constant (K) of 20-80 N/mm along a longitudinal axis of said first anchor.

2. The device of claim 1, wherein said elastically deformable element is positioned within said first anchor.

3. The device of claim 1, wherein said elastically deformable element includes elastically deflectable projections.

4. The device of claim 3, wherein said projections elastically deflect as said elastically deformable element is advanced within said first anchor.

5. The device of claim 3, wherein said deformable element is substantially tube shaped and said projections are longitudinal or vertical.

6. The device of claim 3, wherein said deformable element is substantially disc shaped and said projections are circumferential.

7-8. (canceled)

9. The device of claim 1, wherein said first anchor is an externally threaded hollow tube.

10. The device of claim 9, wherein a first end of said hollow tube includes an external flange.

11. The device of claim 9, wherein a second end of said hollow tube includes an internal bevel.

12. (canceled)

13. (canceled)

14-19. (canceled)

20. A method of interconnecting a first bone region to a second bone region, the method comprising:
(a) positioning a wire between the first bone region and the second bone region;
(b) delivering a first anchor over the wire and into the first bone region;
(c) delivering a second anchor over the wire and into the second bone region;
(d) deforming ends of said wire to thereby trap said ends of said wire against said first anchor and said second anchor.

21. The method of claim 20, wherein (a) is effected by drilling holes through the first bone region and the second bone region using a cannulated drill bit carrying said wire within a lumen thereof.

22. The method of claim 20, wherein said first anchor includes an elastically deformable element and further wherein a deformed end of said wire is trapped against said elastically deformable element.

23. The method of claim 22, further comprising a step of tensioning said wire prior to (d).

24. The method of claim 23, wherein tensioning is effected via a device including a mechanism for engaging and tensioning the wire and a tension gauge for determining a tension on said wire.

25. The method of claim 24, wherein said wire is tensioned to a force of 20-80 N.

26. The method of claim 24, wherein said elastically deformable element has a force constant (K) of 20-80 N/mm along a longitudinal axis of said first anchor.

27. The method of claim 26, wherein the first bone region is in a metatarsal and the second bone region is in an adjacent metatarsal.

28. The method of claim 27, wherein (a) is effected by drilling a straight hole through said metatarsal and said adjacent metatarsal using a cannulated drill bit carrying said wire within a lumen thereof.
